(19) United States
(12) Patent Application Publication
(10) Pub. No.: US 2026/0345980 A1
(43) Pub. Date: Nov. 5, 2026

(54) TATTOO APPLICATOR

(71) Applicant: TAZ TATTOO TECH INC., Sherida, WY (US)

(72) Inventors: Yue WANG, Melbourne (AU); Yi Wu Duan, Mississauga (CA); John Pham, Mississauga (CA)

(21) Appl. No.: 19/537,744

(22) Filed: Feb. 12, 2026

Related U.S. Application Data
(63) Continuation-in-part of application No. 18/410,419, filed on Jan. 11, 2024, now abandoned.

(51) Int. Cl. A61M 37/00

(52) U.S. Cl. CPC A61M 37/0076

(57) ABSTRACT

A disposable tattoo applicator comprising a housing defining an opening at a proximal end of the housing, a two-dimensional array of needles having respective needle tips suitable for penetrating human skin, the two-dimensional array of needles being fixed to or formed integrally with a needle plate and the needle plate being mounted within the housing, a drive mechanism comprising a spring coupled between the housing and the needle plate, and a trigger mechanism that is operable by a user to activate the drive mechanism and thereby apply energy stored in the spring to drive the needle tips into the user's skin, wherein the heights of the needle tips vary across each dimension of the two-dimensional array of needles.

### CROSS-REFERENCE TO RELATED APPLICATIONS

This is a continuation-in-part of U.S. application Ser. No. 18/410,419, filed on Jan. 11, 2024, the entire contents of which being fully incorporated herein by reference.

### TECHNICAL FIELD

The present invention relates to a tattoo applicator and in particular, though not necessarily, to a self-operable, single-use and disposable automatic tattoo applicator.

### BACKGROUND OF THE INVENTION

Existing tattoo applicators for use on human skin typically require a skilled tattoo artist or a cosmetic technician to obtain good results, and nonetheless often cause significant pain and damage to the skin. Furthermore, automatic tattoo applicators that are available are only suitable for applying simple and course tattoo patterns and are complicated to configure and operate. There is, therefore, a desire for a tattoo applicator that is both easy to operate (e.g. by a user on the user's own skin) and capable of producing complex tattoo patterns with no or minimal skin damage.

### SUMMARY OF THE INVENTION

According to a first aspect of the present invention there is provided a disposable tattoo applicator comprising a housing defining an opening at a proximal end of the housing, a set of needles having respective needle tips suitable for penetrating human skin, the needles being fixed to or formed integrally with a needle plate and the needle plate being mounted within the housing, a drive mechanism comprising a spring coupled between the housing and the needle plate, and a trigger mechanism that is operable by the user to activate the drive mechanism and thereby apply energy stored in the spring to drive the needle tips into the user's skin.

In embodiments, the needle tips may be located over a generally convex surface.

In embodiments, the needles may be of uniform length and said needle plate may have a generally convex surface profile.

In embodiments, the needles may have a variety of different lengths, and said needle plate may have a generally flat surface profile.

In embodiments, the disposable tattoo applicator may comprise a sheath axially movable relative to the housing, for example by way of a deployment spring, and configured to move in a proximal direction following penetration of a user's skin by the needle tips to re-cover the needle tips.

In embodiments, the disposable tattoo applicator may be configured to prevent movement of the sheath in a distal direction following re-covering of the needle tips so as to prevent reuse of the applicator.

In embodiments, the needle tips may be configured to provide one or more step changes in height across the generally convex surface.

In embodiments, the spacings between adjacent needle tips in a transverse plane may be varied to provide a desired tattoo pattern following use.

In embodiments, the disposable tattoo applicator may be configured to apply a force to the needle plate following penetration of the skin by the needle tips to retract the needle tips from the skin.

In embodiments, the disposable tattoo applicator may further comprise a mechanism for adjusting the distance that the needle tips protrude from the housing following activation of the drive mechanism.

According to a second aspect of the present invention there is provided a disposable tattoo applicator comprising a housing defining an opening at a proximal end of the housing, a set of needles having respective needle tips suitable for penetrating human skin, the needles being fixed to or formed integrally with a needle plate, and the needle plate being mounted within the housing, and a drive mechanism for pushing the needle plate and the needles through the housing to cause the needle tips to exit the housing and penetrate a user's skin, wherein the needle tips are arranged over a generally convex surface.

In embodiments, the needles may be of uniform length and said needle plate may have a generally convex surface profile.

In embodiments, the needles may have a variety of different lengths, and said needle plate may have a generally flat surface profile.

In embodiments, the needles may have a variety of different angles, and said needle plate may have a generally curved surface profile.

In embodiments, the disposable tattoo applicator may comprise a sheath axially movable relative to the housing, for example by way of a deployment spring, and configured to move in a proximal direction following penetration of a user's skin by the needle tips to re-cover the needle tips.

In embodiments, the applicator may be configured to prevent movement of the sheath in a distal direction following recovering of the needle tips so as to prevent reuse of the applicator.

In embodiments, the needle tips may be configured to provide one or more step changes in height across the generally convex surface.

In embodiments, the spacings between adjacent needle tips in a transverse plane may be varied to provide a desired tattoo pattern following use.

In embodiments, the disposable tattoo applicator further may comprise a mechanism for adjusting the distance that the needle tips protrude from the housing following activation of the drive mechanism.

According to a third aspect of the present invention there is provided a holder configured to accommodate a plurality of adjacent disposable applicators according to the above aspects and comprising a mechanism to activate the respective drive mechanisms of the plurality of disposable applicators substantially simultaneously.

According to a further aspect of the present invention there is provided a disposable tattoo applicator comprising a housing defining an opening at a proximal end of the housing, a two-dimensional array of needles having respective needle tips suitable for penetrating human skin, the two-dimensional array of needles being fixed to or formed integrally with a needle plate and the needle plate being mounted within the housing, a drive mechanism comprising a spring coupled between the housing and the needle plate, and a trigger mechanism that is operable by a user to activate the drive mechanism and thereby apply energy stored in the spring to drive the needle tips into the user's skin, wherein the heights of the needle tips vary across each dimension of the two-dimensional array of needles.

The needle tips may be located over a generally convex or concave surface.

The needles may be of uniform length and said needle plate has a generally convex or concave surface profile.

The needles may have a variety of different lengths, and said needle plate has a generally flat surface profile.

The needle plate may define a three-dimensional profile upon which ends of the two-dimensional array of needles rest, the needles having substantially identical lengths such that the three-dimensional profile is transferred to the needle tips.

Embodiments may comprise a needle cradle located within the housing and configured to be driven by the drive mechanism, the needle plate being fixed to or formed integrally with an end of the cradle, and the cradle defining an inner chamber for holding the two-dimensional array of needle and further defining an opening at an end of the cradle opposed to the needle plate and through which ends of the two-dimensional array of needle, comprising the needle tips, project.

The spring may be in a compressed state and is located between the needle plate and an end of the housing remote from the opening, the trigger mechanism being operable to decouple the needle cradle, and thereby the needle plate, from the housing.

Embodiments may comprise a sealing member covering the opening at a proximal end of the housing, whereby operation of the applicator causes the needle tips to penetrate the sealing member.

Embodiments may comprise a sheath axially movable relative to the housing, for example by way of a deployment spring, and configured to move in a proximal direction following penetration of a user's skin by the needle tips to re-cover the needle tips.

The applicator may be configured to prevent movement of the sheath in a distal direction following re-covering of the needle tips so as to prevent reuse of the applicator.

The spacings between adjacent needle tips in a transverse plane may be varied to provide a desired tattoo pattern following use.

Embodiments may be configured to apply a force to the needle plate in a distal direction following penetration of the skin by the needle tips to retract the needle tips from the skin.

Embodiments may comprise an adjustment mechanism for adjusting a distance that the needle tips protrude from the housing following activation of the drive mechanism.

### BRIEF DESCRIPTION OF THE DRAWINGS

FIG. 1 shows schematically a tattoo applicator according to the present disclosure;

FIG. 2 is a table of photographs of tattoos produced using tattoo applicators having different configurations of needles/needle plates;

FIG. 3 illustrates a substantially flat-ended needle plate;

FIG. 4 illustrates a convex needle plate, with (A) and without (B) needles;

FIG. 5 illustrates a convex needle plate provided with needles of differing lengths;

FIG. 6 illustrates a holder configured to accommodate a plurality of adjacent disposable applicators;

FIGS. 7A-7C illustrate schematically a further tattoo applicator and component parts thereof according to the present disclosure;

FIGS. 8A and 8B illustrate respectively an exemplary core centre part and a corresponding needle tip configuration;

FIG. 9 illustrates an end plate of a cradle of a tattoo applicator with a complex embossed pattern;

FIG. 10 illustrates an alternative tattoo applicator, perspective and axial cross-sectional views, as well as an internal cradle component, embossed pattern design, and needle configuration; and

FIG. 11 shows a needle arrangement with angular orientations, to deliver tattoos in larger areas with more complex designs through one application, based on custom 3D scans of an individual's body regions.

### DETAILED DESCRIPTION OF THE INVENTION

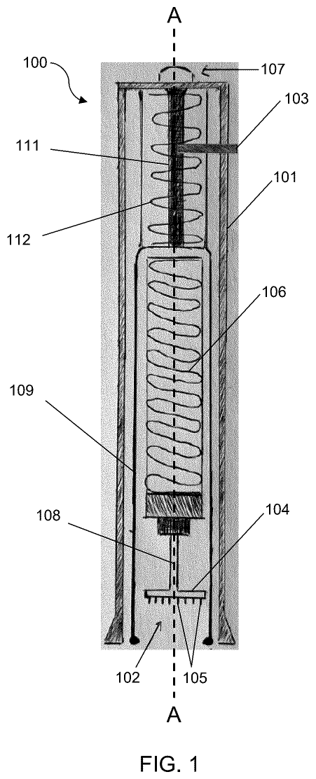

FIG. 1 shows schematically a disposable tattoo applicator 100. The applicator 100 comprises a generally cylindrical outer housing 101 defining an opening 102 at a proximal end of the housing. An inner sheath 109 having a substantially cylindrical shape is concentrically located within the outer housing 101. The sheath 109 is open at its proximal end and is slidably located on an axially extending rod 111 that is fixed to the inside of the distal end of the outer housing 101. A compression spring 112 surrounds the rod 111 between the distal end of the housing 101 and a distal end of the inner sheath 109 such that the spring 112 biases the sheath 109 in a proximal direction.

A circular needle plate 104 is mounted within the inner sheath 109 on a drive shaft 108, and a set of needles 105, configured as a two-dimensional array of substantially aligned needles, are fixed to or formed integrally with the needle plate such that the axes of the needles are aligned with the axis of the outer housing A-A. The needles 105 have respective needle tips suitable for penetrating human skin. The needles 105 are arranged in an array (the array having a diametrical outer extent of, for example, approximately 5 -15 mm), such that the needle tips form a pattern corresponding to a desired tattoo design. Suitable tattoo designs include shapes (e.g. circles or hearts) or letters/glyphs or complex designs (e.g. “Mona Lisa”). The set of needles may comprise any suitable number of needles, although the approach described here lends itself to the application of complex and fine-detailed patterns potentially requiring a large number of needles, for example hundreds or even thousands of needles.

A drive spring 106 in the form of a compression spring acts between an inner surface of a distal end of the inner sheath 109 and the drive shaft 108 and needle plate 104. In a pre-use state the drive spring 106 is in a compressed state and latched by a latching mechanism to retain it in this compressed state. The latching mechanism is releasable by activation of a trigger 107 that projects from an outer surface of a distal end of the outer housing. The trigger 107 may, for example, when operated act through the rod 111 to release the latching mechanism.

A safety mechanism is also provided in the form of a “pin” 103, to prevent the applicator from being misfired (e.g. during shipping, or accidentally by the user). When in place, the pin 103 extends through a cut-out portion in the outer housing 101 and prevents the release of the drive spring 106 (e.g. by preventing the rod 111 from moving when a force is applied to the trigger 107). When the applicator is ready for use, the pin 103 is removed by the user to allow the applicator to fire.

In the pre-use state the inner sheath 109 is pushed by the compression spring 112 such that the end of the sheath projects out of the proximal end of the outer housing 101, e.g. by 1-5 mm. The drive spring 106 is held in a compressed state such that the needle plate 104 remains within the inner sheath thereby avoiding risk of needle stick injuries.

Tattoo ink may be applied to the needle tips of the present applicator in a variety of different ways. In some examples, the applicator 100 comprises a built-in tattoo ink reservoir (not shown). For example, an ink pouch may be located within the housing of the applicator, such that the needle tips are pushed through the pouch upon activation of the drive mechanism. Alternatively, ink can be provided in a tube (e.g. located on the drive shaft 108), wherein the tube is configured to release the ink upon activation of the drive mechanism, whereupon the ink drips onto the needle tips below.

Alternatively, the ink may be provided externally to the applicator. For example, before use, the user may apply tattoo ink to the needles 105 by dipping the needles into an ink reservoir that is separate from the applicator. Alternatively, liquid ink can be applied directly to the user's skin. The applicator is then placed and activated directly over the applied ink, and the excess ink on the surface of the skin is wiped away after use. Ink can also be directly applied onto the user's skin using “dry” (semi-wet) ink patches or other sticker methods, then tattooed using the applicator. Similarly, the ink may be impregnated within a layer of paper or the like, and the paper laid over the skin such that ink is pressed from the paper into the skin. The present device is suitable for use with tattoo inks that are multi-coloured, single-coloured, permanent or temporary.

During use, the user grasps the outer housing in his or her fist and places the end of the applicator onto the ink, paper etc, so that the proximal end of the inner sheath is in contact with the skin, whilst exerting a force against the skin to compress the compression spring 112 and push the inner sheath into the outer housing until the proximal end of the inner sheath is substantially flush with the proximal end of the outer housing. This is the state shown in FIG. 1 and is intended to achieve a consistent force against the skin upon insertion of the needles. The user then activates the drive mechanism by operating the trigger 107, causing the needle tips to penetrate the user's skin and to push ink into the skin (e.g. between the epidermis and the dermis) to automatically form a tattoo according to the design. NB. An interlock may be provided between the inner sheath and the trigger such that the trigger can only be operated when the inner sheath is fully within the outer housing.

Upon activation of the trigger 107, the drive spring 106 is released, allowing it to expand within the inner sheath 109, thereby driving the drive shaft 108 and attached needle plate 104 through the inner sheath until the needle tips contact the skin and push through the outer layer of the skin. The applicator is configured such that the needle tips exert sufficient force on the skin to perform the desired function. The user can then remove the applicator from the skin whereupon the compression spring 112 is free to expand.

In some examples, the applicator 100 includes a “bounce-back” mechanism that is configured to apply a force to the needle plate following penetration of the skin by the needle tips, in order to actively retract the needle tips from the skin. This may enhance the deposition of the ink into the skin by creating a region of low pressure in the channels left behind by the needle tips, which draws the ink from the needles into the skin. The bounce-back mechanism also means that the needles are less likely to remain in the skin after use, reducing the likelihood that the user needs to pull the needle plate out of his/her skin. This both reduces the risk of injury and renders the process more comfortable for the user (especially for users with needle phobias, for example). The bounce-back force may be provided by the spring 112 (e.g. as a result of over-extension following activation of the drive mechanism), or by one or more dedicated “return” springs additionally located within the housing 101 (e.g. that become compressed between a portion of the housing and the needle plate following activation of the drive spring 106). It will also be appreciated that at the end of its extension range, the drive spring 106 will be slightly over-extended, which may result in some “bounce-back” of the needle tips.

It will be appreciated that, following use of the described applicator, the needle tips may project outwardly from the end of the applicator. The skilled person will appreciate that a mechanism may be included in the device to enable a re-covering or re-sheathing of the needles following use. This may be automatic or manually operated. For example, an additional outer sheath may surround the proximal end of the outer housing, being coupled thereto by a spring that is compressed when the outer sheath is pressed against the skin and expand to re-cover the needle tips on removal of the applicator from the skin.

A simplified design may be employed involving a main housing, a compression spring, a drive shaft and a needle plate, with a latching and trigger mechanism for holding the needle plate in a retracted position and allowing its release to drive the needle tips forward and into the skin. It will also be appreciated that drive means other than a compression spring may be used, for example a torsion spring.

According to conventional tattooing methods, needles from manually operated tattoo guns break the skin multiple times during multiple passes by the tattoo artist, resulting in significant and unevenly distributed damage to the skin. This results in skin “peeling”, during a healing process which usually lasts 1-2 weeks. By pre-arranging the needles 105 on the needle plate 104 in an array according to the desired tattoo design, and then using the drive mechanism to push the pre-arranged needle tips into the skin, the present applicator 100 is able to produce a tattoo with improved efficiency and accuracy, and with less damage and trauma to the skin, resulting in a faster healing process. The resulting tattoos can also be easily and quickly applied by a user to the user's own skin.

Furthermore, the present applicator allows for the needle tips to automatically penetrate the skin to a consistently defined depth. This limits the opportunity for human error resulting in the needles penetrating too deeply into the skin (e.g. below the dermis layer, which is present at a depth of around 1-4 mm on average), which can otherwise cause so-called “blow-outs” (when ink is deposited between the dermis and hypodermis/fat layers) and “bumps” (when ink is deposited into the hypodermis/fat layer).

In some examples, the applicator further comprises an adjustment mechanism for adjusting the distance that the needle tips protrude from the housing following activation of the drive mechanism. For example, the position of the needle plate 104 within the housing 101 prior to the activation of the drive mechanism may be adjustable, e.g. by rotating the trigger 107 (in a twisting action, in the manner of a watch crown) to adjust a position of the rod 111 in the proximal direction using cooperating threads provided on the rod 111 and the housing 101. By providing a mechanism for adjusting the distance that the needle tips protrude from the housing during use, a single applicator design can be used to effectively tattoo different areas of the body having different skin thicknesses.

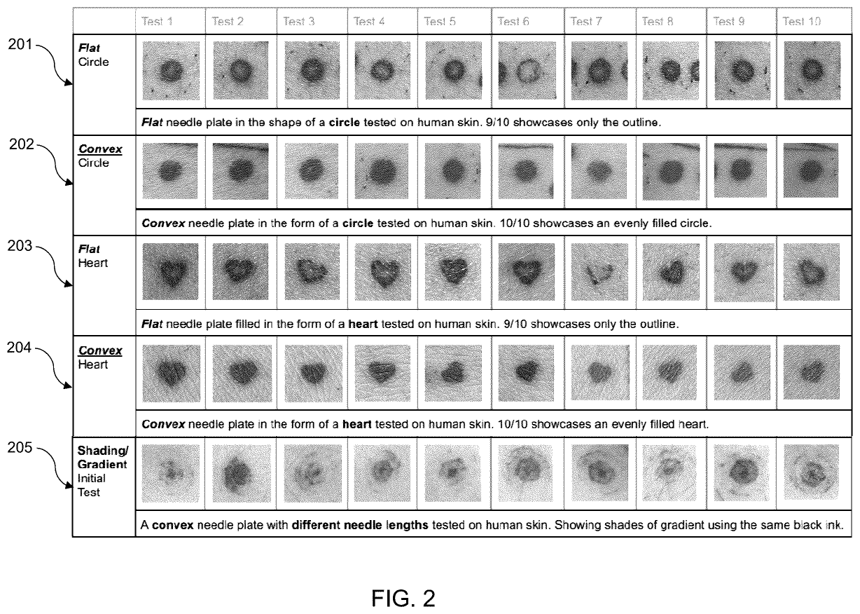

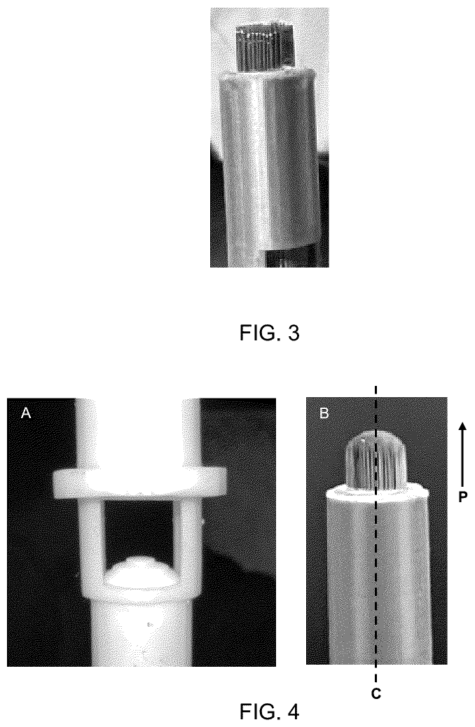

Applicators comprising different configurations of needles and needle plates have been tested on human skin, and the results are shown in FIG. 2. The testing demonstrated that, when a flat-ended needle arrangement, such as is illustrated in FIG. 3, hits the skin at high speed (due to the potential energy released from the loaded spring), it deforms the skin surface with a substantially concave profile. This appears to be due to the elasticity of the skin. This results in a tattoo with a poorly defined outline and uneven filling (as shown in rows 201 and 203 of FIG. 2).

To counteract this effect, the needle tip arrangement of the present applicator may be provided with an overall generally convex profile, as shown in FIG. 4. That is, an extent of the needle tips in the proximal direction P (i.e. in the direction of the skin during use) is generally greater for needles that are positioned closer to the central axis C of the needle plate than for needles that are positioned closer to the perimeter of the needle plate. This arrangement allows the needle tips to follow the concave profile formed in the skin. Panel A shows a needle plate with a convex profile (without needles), and Panel B shows the convex profile of the needle tips, resulting from the convex profile of the needle plate (i.e. with needles of the same length). As shown in FIG. 2, rows 202 and 204, by using a convex needle plate, the problem of uneven filling is resolved or at least mitigated. It is noted that, whilst following a generally convex profile, the needle tip arrangement may include intermediate step changes in height, e.g. with two or more steps.

Without wishing to be bound by theory, the optimal convex curvature of the needle tips/needle plate can be mathematically calculated using the Poisson ratio for human skin of average elasticity. Human skin has an expansion/contraction ratio of around 0.48 at maximum force before breaking. In practice, the number should be around 0.3, lower than the breaking point. The optimal stretch curve can be calculated based on the diameter of the needle plate (or width, if it is an irregular shape), and the force generated by the spring.

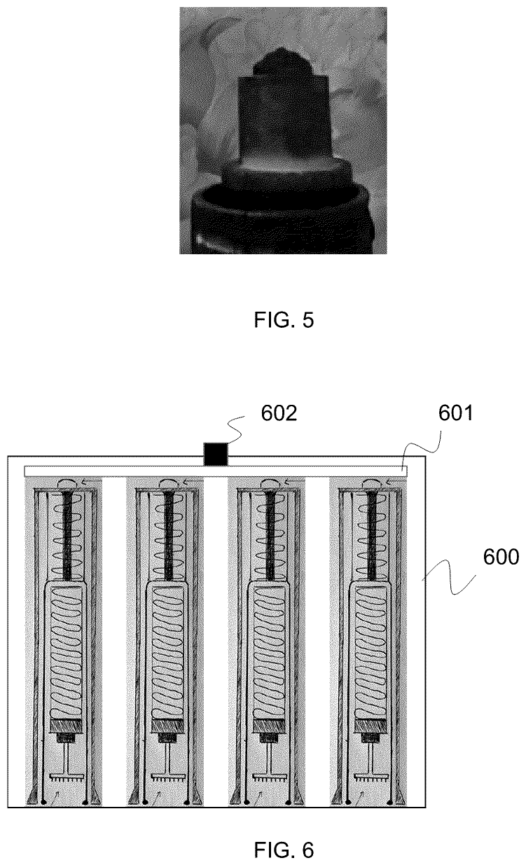

In some examples, a convex needle plate can be used in conjunction with needles having different lengths, as shown in FIG. 5. This allows for ink to be deposited into the user's skin at various depths, resulting in a tattoo with multiple tones. In this way, the applicator can deliver an instant tattoo with gradients and shading, as shown in row 205 of FIG. 2.

In some cases, a concave needle tip surface profile may be appropriate, for example obtained by means of a concave needle plate with needles of identical length. A concave profile may be better suited when applying a tattoo to an ear of skin with a complimentary profile.

The needles can also be provided at different densities in different regions of the needle plate, corresponding to different regions of the tattoo design. By adjusting the needle “resolution” in this way, alternative or additional shading effects can be produced.

Multiple tattoo applicators as described herein may be provided within a single holder, and arranged within the holder so as to produce a single tattoo on the user's skin having (e.g. a complex design) when the multiple applicators are fired simultaneously. For example, a plurality of applicators, each having an array of needle tips arranged in the form of a letter, may be arranged within the holder and activated collectively on the user's skin to produce a tattoo in the form of a word. The holder may comprise a mechanism for activating the respective drive mechanisms of the plurality of applicators substantially simultaneously. FIG. 6 illustrates an exemplary holder comprising a main housing 600 configured to hold four applicators arranged in a row. The holder comprises a common firing mechanism 601 with a trigger 602 such that all applicators can be fired simultaneously.

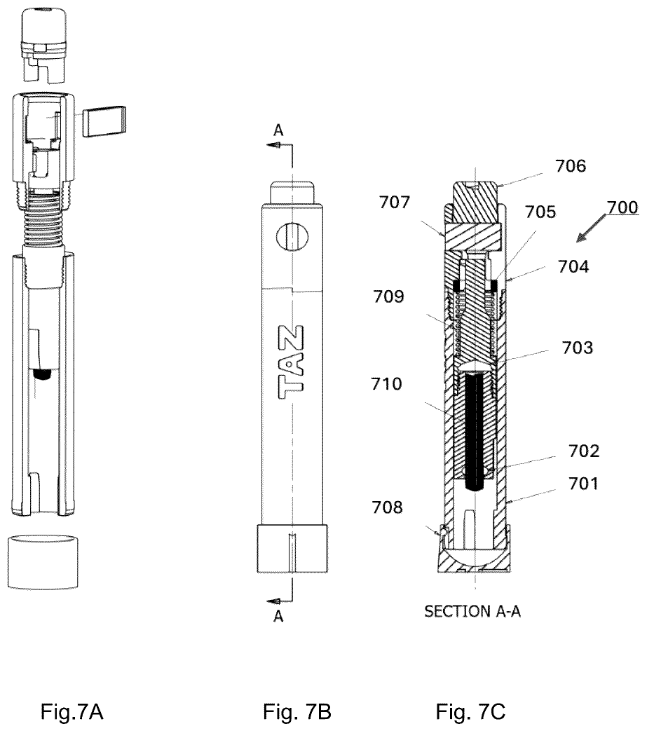

FIG. 7A illustrates schematically an exploded view of an alternative tattoo applicator apparatus 700, whilst FIG. 7B illustrates schematically the apparatus in an assembled, pre-firing state. FIG. 7C illustrates an axial cross-section of the assembled apparatus of FIG. 7B.

The operating principle of the embodiment of FIGS. 7A-7C is similar to that of the previously described embodiment. The following component parts are particularly pointed out:
Main casing 701
Needle cradle 702
Centre Core 703
Top stage 704
Inner trigger 705
Outer trigger 706
Safety key 707
Bottom cap 708
Compressed drive spring 709
Needle set 710

The needle cradle 702 may be sized internally to enable a tight packing of the needles within the cradle, whilst allowing some degree of axial movement along the cradle, at least during packing of the needles into the cradle.

In order to operate the applicator, a user first removes the safety key 707. This then allows the user to depress the outer trigger 706 with his or her thumb which in turn acts on the inner trigger 705 to decouple the needle cradle 702 from the main housing 701. This allows the drive spring 708 to expand, pushing the needle cradle 702 and the contained set of needles 710 down through the housing. The needle tips penetrate through a sealing membrane closing the end of bottom cap 708 and bring them into contact with the user's skin. Ink (pre) applied to the tips is then introduced into the skin.

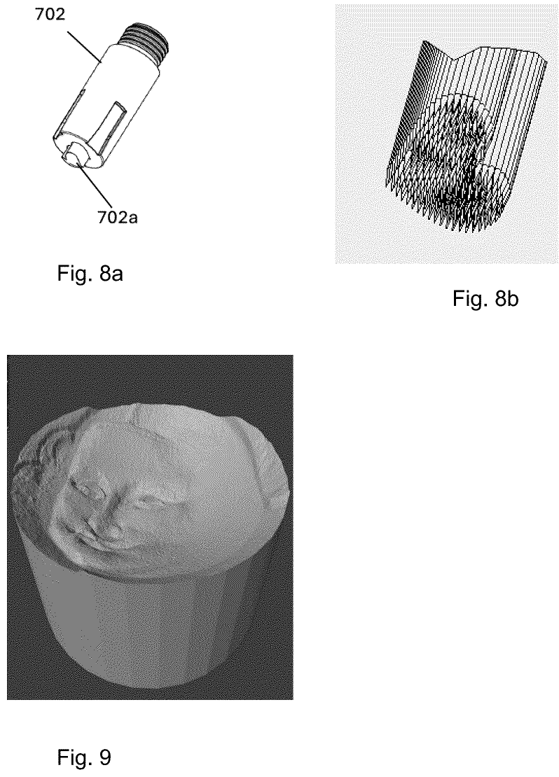

FIG. 8A illustrates in greater detail an end perspective view of the needle cradle 702 and which contains the set of needles. The cradle is substantially hollow to accommodate the needles and has a pattern embossed on an and surface 702a. In this example the pattern is a recessed heart shape. The pattern produces a complimentary profile on an inner surface of the end of the cradle such that, when applied to a user's skin, the pattern will be a negative image of the heart. Within the cradle, the needles, which all have the same length, locate against the inner surfaces and the profile such that the embossed pattern is established across the needle tips. The needle tip configuration is shown in FIG. 8B.

It will be appreciated that the illustrated embodiment allows for different needle cradles with different embossed patterns to be used. The cradles may be 3D-printed, allowing for example the creation of bespoke patterns for users. FIG. 9 illustrates a more complex embossed pattern, in this case the “Mona Lisa”.

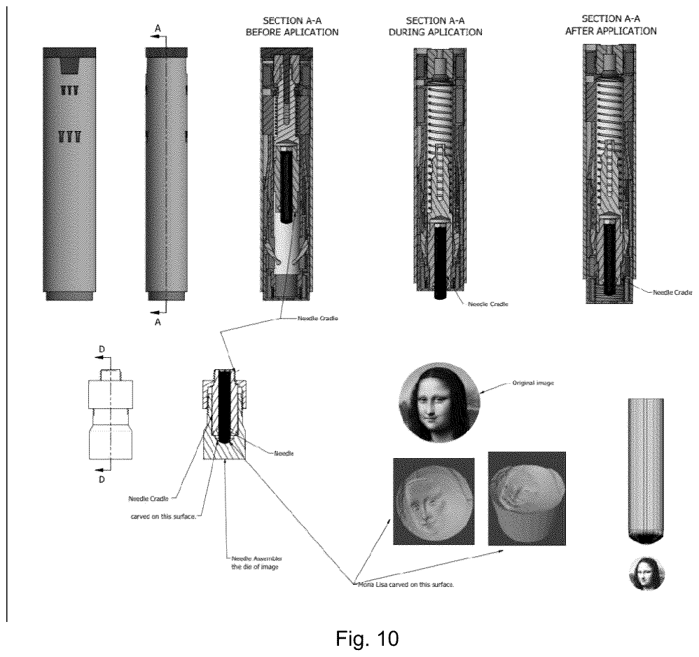

FIG. 10 illustrates a further modified embodiment which provides for deployment of a needle shield through a lower opening of the housing following application. As can be seen in the drawings, deployment of the shield causes the needle tips to be covered, significantly reducing the risk of needle-stick injury after use.

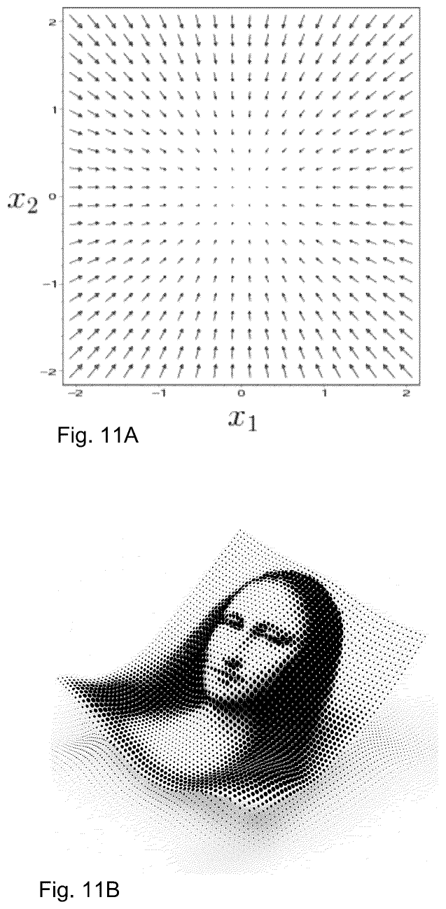

The skilled person will appreciate that a number of modifications can be made to the applicator/holder as described herein. For example, whilst the above description relates to a spring-loaded drive mechanism, other suitable manually loaded mechanisms are also contemplated (e.g. electrical or magnetic based mechanisms). In one example, the applicator, or holder, may be electronically operated by the user through a computer device (e.g. a desktop computer, laptop, phone, tablet, headset or the like) connected to the applicator wirelessly or via a wired connection), for example at the press of a key (keyboard), a touch interface (touchscreen) or a spatial AR interface. In addition, whilst the above discussion relates to the use of a convex needle plate, it will be appreciated that a convex pattern of the needle tips can be achieved using a flat needle plate and needles of varying lengths. In a still further modification of the described embodiments, all or some of the needles may be arranged at an angle to the main axis of the device. This is illustrated in FIGS. 11A and 11B, where the formed is an end view of the need (tip) array and the latter illustrates the produced design. In some cases, the arrangement of the needles, including the angular orientations, may be determined from a 3D scan of a skin region to which the tattoo is to be applied. This may be a bespoke scan of a given individual to whom the tattoo is to be applied, or may be a scan of a generic individual.

1. A disposable tattoo applicator comprising:
a housing defining an opening at a proximal end of the housing;
a two-dimensional array of needles having respective needle tips suitable for penetrating human skin, the two-dimensional array of needles being fixed to or formed integrally with a needle plate and the needle plate being mounted within the housing;
a drive mechanism comprising a spring coupled between the housing and the needle plate; and
a trigger mechanism that is operable by a user to activate the drive mechanism and thereby apply energy stored in the spring to drive the needle tips into the user's skin,
wherein the heights of the needle tips vary across each dimension of the two-dimensional array of needles.

2. The disposable tattoo applicator of claim 1, wherein the needle tips are located over a generally convex or concave surface.

3. The disposable tattoo applicator of claim 2, wherein the needles are of uniform length and said needle plate has a generally convex or concave surface profile.

4. The disposable tattoo applicator of claim 2, wherein the needles have a variety of different lengths, and said needle plate has a generally flat surface profile.

5. The disposable tattoo applicator of claim 1, wherein said needle plate defines a three-dimensional profile upon which ends of the two-dimensional array of needles rest, the needles having substantially identical lengths such that the three-dimensional profile is transferred to the needle tips.

6. The disposable tattoo applicator of claim 5 and comprising a needle cradle located within the housing and configured to be driven by the drive mechanism, the needle plate being fixed to or formed integrally with an end of the cradle, and the cradle defining an inner chamber for holding the two-dimensional array of needle and further defining an opening at an end of the cradle opposed to the needle plate and through which ends of the two-dimensional array of needle, comprising the needle tips, project.

7. The disposable tattoo applicator of claim 5, wherein the spring is in a compressed state and is located between the needle plate and an end of the housing remote from the opening, the trigger mechanism being operable to decouple the needle cradle, and thereby the needle plate, from the housing.

8. The disposable tattoo applicator of claim 1 and comprising a sealing member covering the opening at a proximal end of the housing, whereby operation of the applicator causes the needle tips to penetrate the sealing member.

9. The disposable tattoo applicator of claim 1 and comprising a sheath axially movable relative to the housing, for example by way of a deployment spring, and configured to move in a proximal direction following penetration of a user's skin by the needle tips to re-cover the needle tips.

10. The disposable tattoo applicator of claim 8, the applicator being configured to prevent movement of the sheath in a distal direction following re-covering of the needle tips so as to prevent reuse of the applicator.

11. The disposable tattoo applicator of claim 1, wherein the spacings between adjacent needle tips in a transverse plane is varied to provide a desired tattoo pattern following use.

12. The disposable tattoo applicator of claim 1 and configured to apply a force to the needle plate in a distal direction following penetration of the skin by the needle tips to retract the needle tips from the skin.

13. The disposable tattoo applicator of claim 1 and comprising an adjustment mechanism for adjusting a distance that the needle tips protrude from the housing following activation of the drive mechanism.

14. The disposable tattoo applicator of claim 1, wherein the two-dimensional array of needles comprises at least 100 needles, preferably more than 1000 needles.

15. The disposable tattoo applicator of claim 1, wherein all or some of the needles are arranged at an angle to the main axis of the device.
